(19) United States
(12) Patent Application Publication
(10) Pub. No.: US 2004/0219176 A1
(43) Pub. Date: Nov. 4, 2004

(54) Injectables in foam. new pharmaceutical applications

(76) Inventor: Maria Antonia Garcia-Olmedo Dominguez, Granada (ES)

Correspondence Address: KLAUBER & JACKSON, 4th Floor, 411 Hackensack Avenue, Hackensack, NJ, 07601, US

(21) Appl. No.: 10/774,071

(22) Filed: Feb. 6, 2004

Related U.S. Application Data
(63) Continuation of application No. PCT/ES01/00317, filed on Aug. 8, 2001.

(51) Int. Cl. A61K 9/00

(52) U.S. Cl. 424/400

(57) ABSTRACT

The placing of a drug that is suitable for parenteral administration on bubbles formed with sterile gases produces an exponential increase in its active surface area with a decrease in the diameter of the bubble, modifies the kinetics of its distribution and, thanks to its micronisation, increases its therapeutic effect. Furthermore, the echogenicity of the bubbles allows us to follow them on ultrasound after their injection, so that we can visualise the medicament and, thanks to its steerability, can direct it to the selected site or prevent it from reaching undesired areas. This pharmaceutical form is of interest in the treatment of diseases that require a greater local action of the injected drugs than can be achieved with the pharmaceutical forms in current use.

### RELATED APPLICATIONS

The present application is a Continuation of co-pending PCT Application No. PCT/ES01/00317, filed Aug. 8, 2001. Applicant claims the benefits of 35 U.S.C. §120 as to the PCT application, and the entire disclosure of the application is incorporated herein by reference in its entirety.

### INTRODUCTION

Parenteral administration is carried out through the skin barrier, in order to introduce medicaments in tissues or organ cavities that are not directly communicated with the exterior and even to introduce them directly into the blood stream, which acts as a distribution system.

One benefit of injectables is their rapid action (almost instantaneous in intravenous administration because the medicinal substances are immediately distributed by the blood) but it can be difficult to maintain this action locally for a given time, above all in highly vascularised areas. Furthermore, it can be laborious to achieve therapeutic concentrations of a substance in poorly vascularised areas or at sites where the traditional distribution via the bloodstream is inadequate to achieve the necessary concentrations of a medicament or to retain the medicament in the selected area for the requisite time for its action to have effect.

With the drug delivery system of injectable foam we achieve a more prolonged local action, also in richly vascularised organs and even in blood vessels. Moreover, the steerability of this pharmaceutical form makes it difficult for the action to reach undesired zones.

Furthermore, the micronisation of the medicinal substances that is produced when we place them on bubbles exponentially increases their active surface area, with the result that the same therapeutic effect is achieved with lower doses. Another benefit conferred by this pharmaceutical form is the possibility of observing ultrasonographically where the medicament is sited.

This drug delivery system may be of interest in the treatment of multiple diseases where the local action of the drugs and medicinal substances injected is of value and cannot be achieved with the pharmaceutical forms in current use.

### STATE OF THE PREVIOUS TECHNIQUE

According to patent EP,A, 0 077 752 (SCHERING AKTIENGESELLSCHAFT) 1983, liquid mixtures with physiologically compatible gas bubbles have been used as a contrast medium in ultrasound diagnosis.

There have also been attempts, according to patent WO,A, 92 05806 (SINTÉTICA S. A.) 1992, to obtain more stable suspensions of microspheres filled with gas in aqueous liquids, suitable for injection as a medium to increase the echogenicity of the blood and reinforce the ability of ultrasonography to aid medical diagnoses, as for example in the detection of vascular diseases.

Microfoam containing sclerosants has also been injected for the treatment of varicose veins and outcomes have been observed to be superior to those obtained with liquid sclerosants. (WO 95/00120 J. CABRERA GARRIDO, 1995).

### PREPARATION

This invention refers to the preparation of an injectable foam with any medicinal substance, adding foaming agents and gases and producing it in accordance with the conditions required.

In some diseases to be treated, the therapeutic agent can be the gas used in the formation of the foam.

The foam can be produced A) by mechanical or ultrasonic whisking of the solution, B) by depressurisation of a solution that incorporates gas dissolved under pressure, C) after the release of a gas contained in a compartment that is independent of the solution to be foamed and that is released and placed in contact with the solution at the moment of its use, D) through a chemical reaction that produces the gas, etc.

In patents U.S. Pat. No. 4,446,442, EP-A-131 540, U.S. Pat. No. 4,276,885, procedures are revealed to manufacture solutions of microcapsules or hollow microparticles filled with gas, i.e., microspheres in which the gas is strictly encapsulated. These procedures seek a stability of the microspheres once they are injected into the blood, which allows them to resist their destruction on their intravascular journey and thus to be detected by ultrasonography in vessels that are distant from the injection site. The high stability of these suspensions of microspheres is a necessary condition for their diagnostic efficacy.

Our invention is not attempting to achieve this, but rather to transform into foam any medicinal substance in the presence of gases and foaming agents, but without this producing a dispersion of the microbubbles, which, by continuing to be united by an immaterial boundary, form a different physicochemical entity from solutions of microparticles.

Optionally, it may be of value to improve the cohesion between the bubbles with rheologic agents.

### APPLICATIONS

Injectable foam is of utility, among other cases, in hepatic or renal insufficiency, or in the administration of drugs with little therapeutic margin, such as cytostatics, where we wish to achieve the maximum efficacy of the medicaments with the lowest possible dose delivered as closely as possible to the target tissue.

In localised tumours, the injection of antiinflammatories or corticosteroids in foam may reduce the gastrointestinal risks produced by these agents when they are systemically administered.

In the same way, foam is beneficial in the intravenous use of medicinal substances, for example to promote the local vasodilatation of an ischaemic foot, facilitating the continuance of the injected drug in this zone for the longest possible time.

In abscesses or localised infections, we achieve with injectable foam a more prolonged action of antibiotics or chemotherapeutic antiviral agents in situ, rendering them more efficacious than when they are administered traditionally.

In cases of tinea unguium, given the difficulty of achieving a satisfactory action with systemic administration, it may be of interest to inject beneath the nail foam that contains antimycotic agents.

Another application of injectable foam may be in local anaesthesia, facilitating the diffusion or delaying the distribution of the anaesthetic and thus reducing the repetition of doses.

Moreover, when the therapeutic agent is a gas, it can be maintained in contact at the required site, formed into an injectable foam with inert substances. This would be the case of the administration of oxygen in gas gangrene produced by anaerobic germs or in severe ischaemia of the extremities.

The foam may also be of special utility when the blood cannot be the transport vehicle of a medicament and when an especially intense or selective local action is required, e.g., the in situ use of fibrinolytics/thrombolytics at an adequate concentration in the centre of a thrombosis of an important venous trunk.

To summarise, when it is necessary to maintain the action of an injectable medicament in a given territory, the foam form can provide an increase in its local therapeutic activity, in function of the longer time of its presence, of the reduced dilution at the site required and of the greater active surface area of the medicament.

1. PHARMACEUTICAL FORM OF INJECTABLE FOAM, both already prepared and as an extemporaneous preparation, characterized to include any medicinal substance or drug other than sclerosing agents as well as FOAM FOR INJECTABLES formed with inert foaming agents and any gas.

2. INJECTABLE FOAM, both already prepared and as an extemporaneous preparation, in accordance with claim 1, characterized in that the medicinal substance is a vasodilator.

3. INJECTABLE FOAM, both already prepared and as an extemporaneous preparation, in accordance with claim 1, characterized in that the medicinal substance acts on the cardiovascular system.

4. INJECTABLE FOAM, both already prepared and as an extemporaneous preparation, in accordance with claim 1, characterized in that the medicinal substance is an antimycotic.

5. INJECTABLE FOAM, both already prepared and as an extemporaneous preparation, in accordance with claim 1, characterized in that the medicinal substance is an anti-infectious agent.

6. INJECTABLE FOAM, both already prepared and as an extemporaneous preparation, in accordance with claim 1, characterized in that the medicinal substance is an antibiotic including trimethoprim.

7. INJECTABLE FOAM, both already prepared and as an extemporaneous preparation, in accordance with claim 1, characterized in that the medicinal substance is any other antibiotic or chemotherapeutic agent.

8. INJECTABLE FOAM, both already prepared and as an extemporaneous preparation, in accordance with claim 1, characterized in that the medicinal substance is a sulphonamide.

9. INJECTABLE FOAM, both already prepared and as an extemporaneous preparation, in accordance with claim 1, characterized in that the medicinal substance is a cytostatic.

10. INJECTABLE FOAM, both already prepared and as an extemporaneous preparation, in accordance with claim 1, characterized in that the medicinal substance is an anaesthetic.

11. INJECTABLE FOAM, both already prepared and as an extemporaneous preparation, in accordance with claim 1, characterized in that the medicinal substance is an anti-inflammatory.

12. INJECTABLE FOAM, both already prepared and as an extemporaneous preparation, in accordance with claim 1, characterized in that the medicinal substance is a prostaglandin.

13. INJECTABLE FOAM, both already prepared and as an extemporaneous preparation, in accordance with claim 1, characterized in that the medicinal substance is a corticosteroid.

14. INJECTABLE FOAM, both already prepared and as an extemporaneous preparation, in accordance with claim 1, characterized in that the medicinal substance has hormonal action.

15. INJECTABLE FOAM, both already prepared and as an extemporaneous preparation, in accordance with claim 1, characterized in that the medicinal substance is an antiviral agent.

16. INJECTABLE FOAM, both already prepared and as an extemporaneous preparation, in accordance with claim 1, characterized in that it is formed with inert foaming agents and any gas.
